(19) United States
(12) Patent Application Publication
(10) Pub. No.: US 2009/0090623 A1
(43) Pub. Date: Apr. 9, 2009

(54) Biosensor having integrated heating element and electrode with metallic catalyst

(75) Inventors: Min-Chieh Chuang, Taoyuan Hsien (TW); Yun-Chu Ho, Taoyuan Hsien (TW); Tai-Kang Shing, Taoyuan Hsien (TW); Chung-Chiun Liu, Cleveland Heights, OH (US); Jie Shen, Cleveland, OH (US)

Correspondence Address: BIRCH STEWART KOLASCH & BIRCH, PO BOX 747, FALLS CHURCH, VA, 22040-0747, US

(73) Assignee: DELTA ELECTRONICS, INC., Taoyuan County (TW)

(21) Appl. No.: 12/153,272

(22) Filed: May 15, 2008

Related U.S. Application Data
(60) Provisional application No. 60/924,552, filed on May 21, 2007.

(51) Int. Cl. G01N 33/50; H01B 1/22

(52) U.S. Cl. 204/403.01; 204/403.15; 252/506; 252/503

(57) ABSTRACT

The invention discloses a biosensor includes a strip having an integrated heating element and an electrode with metallized graphite including metal in the range of 0.1-5% in weight; graphite in the range of below 55% in weight; and polymer. The biosensor further includes an electric measuring device having a slot enabling the strip to insert therein.

The present application claims priority of U.S. provisional application Ser. No. 60/924,552, filed May 21, 2007, the entire disclosures of which are hereby incorporated by reference therein.

### BACKGROUND OF THE INVENTION

1. Field of the Invention

The invention relates to a biosensor and, in particular, to a biosensor having an integrated heating element and an electrode with metallized graphite.

The invention will be described to a biosensor for measuring the concentration of glucose in blood but is not limited to that use and has general application for measuring the concentration of an analyte in solution other than blood samples.

2. Related Art

The conventional biosensor for measuring glucose in blood includes a test strip and an electric measuring device. Electrodes are disposed on the surface of the strip and enzyme is disposed on a restricted area of the strip. A user inserts the strip to the electric measuring device and then drops the sampled blood on the restricted area of the strip. The glucose in the blood can be sensed electrochemically to produce a current signal which can be interpreted to give an estimate of the glucose concentration in the blood sample.

The conventional composition for preparing the electrochemical sensing electrode includes a conductive material and an organic solvent. Generally, the substrate of the strip is made of polyvinyl chloride (PVC) or polyester and the composition is disposed on the substrate by screen printing. The electrode dispose on the strip is formed after drying the printed composition. The electrode has a better adhesion with the substrate since the composition is dispersed by organic solvent. However, the electrode made by the conventional composition is unfavorable to measure the concentration of an analyte in a biological sample in aqueous solution since higher impedance arises in the interface between the hydrophobic electrode and the biological sample such as blood.

Another conventional composition for preparing electrode disposed on the strip includes inorganic solvent. The drawback of the electrode made by the composition including inorganic solvent is the undesired adhesion between the electrode and the strip. Furthermore, the electrode made by the composition including inorganic solvent is easier to be destroyed by the sample in aqueous form so that the result of the concentration of the analyte is influenced.

Furthermore, in a common method for measuring the concentration of glucose, the blood sample is reacted with an enzyme for example glucose oxidase or glucose dehydrogenase (GDH). The reaction is easily influenced under the ambient temperature so that the accuracy of the result is unfavorable.

### SUMMARY OF THE INVENTION

The invention discloses a biosensor and a biosensor having an integrated heating element and an electrode with metallized graphite. The concentration of an analyte, for example glucose in blood sample, can be measured to produce the result much efficiently and accurately.

A biosensor according to the present invention for measuring biological analyte, for example glucose in blood, includes a test strip and an electric measuring device. The electric measuring device includes a slot enabling the strip to insert therein. The strip includes a substrate and at least two electrodes disposed on the strip to define a sample area. A reagent, including enzyme, is disposed on the sample area so that an electrochemical reaction can be taken place thereon by the enzyme and glucose in the blood.

According to one aspect of the present invention, the electrode is made of a conductive composition including metallized graphite in the range of 5-30% in weight; polymer in the range of 5-20% in weight; and inorganic solvent. The metallized graphite includes nano-sized metal particle coated on the surface of the graphite particle. The nano-sized metal particle is a catalyst of the electrochemical reaction such as oxidation or reduction of hydrogen peroxide, or oxidation of nicotinamide adenine dinucleotide (NADH). The oxidation or reduction potential can be reduced by the nano-sized metal particle.

The conductive composition is dispersed by inorganic solvent so that the higher impedance can be prevented during the electrochemical reaction. The polymer includes a binder and a water-strength polymer so that the electrode can prevent to be destroyed in the aqueous sample. Furthermore, a conductive layer can be disposed between the substrate and the electrode so that the adhesion between the substrate and the electrode is maintained. The conductive layer includes graphite dispersed in an organic solvent.

According to another aspect of the present invention, the strip further includes a heating element disposed on the substrate and corresponding to the sample area. The sample in the sample area can be heated by the heating element at an elevated or steady temperature depending to the variety of enzyme of the reagent so that the electrochemical reaction can be enhanced.

According to the other aspect of the present invention, the slot of the electrical measuring device includes several pins corresponding to the electrodes and the heating element on the strip. The strip can be electrically connected to the electric measuring device by the pins connected to the electrode and the heating element. Therefore, the electric measuring device is capable for recognizing the insertion of the strip to the slot by electrical connection of the pins and the heating element.

Further scope of the applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given herein below and the accompanying drawings which are given by way of illustration only, and thus are not limitative of the present invention, and wherein:

FIG. 1 shows an exploded view of a strip according to an embodiment of the present invention;

FIG. 2 shows an exploded view of a variation of the strip shown in FIG. 1; and

FIG. 3 shows a schematic representation of an electric measuring device according to the embodiment of the present invention.

### DETAILED DESCRIPTION OF THE INVENTION

The present invention will be appeared from the following detailed description, which proceeds with reference to the accompanying drawings, wherein the same references relate to the same elements.

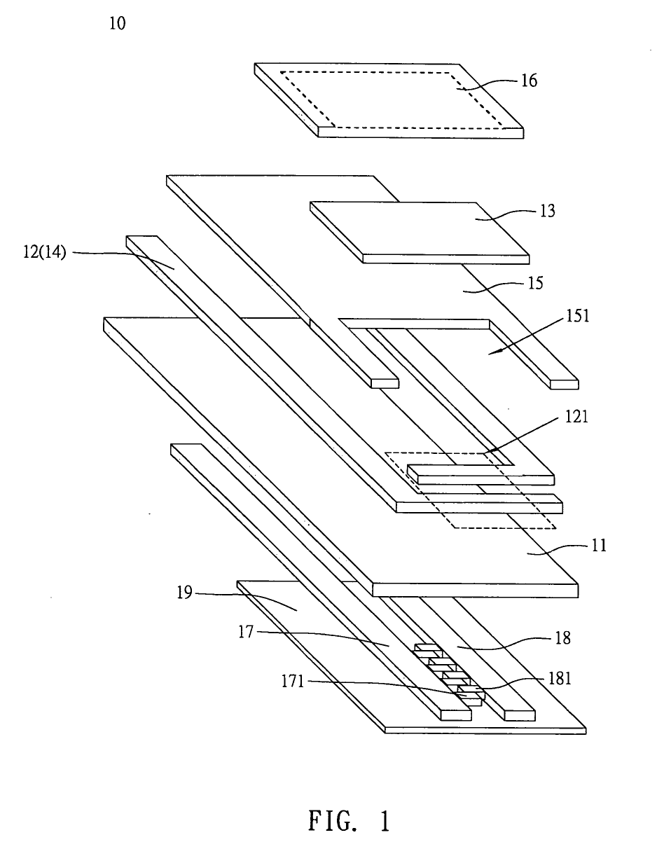

The first feature of the present invention is the electrode includes metallized graphite. As shown in FIG. 1, a biosensor according to an embodiment of the present invention includes a strip 10 having a substrate 11; at least two electrodes 12 disposed on the substrate 11 to define a sample area 121; and a reagent 13 disposed on the sample area 121. The blood sample is dropped to the sample area 121 so that the electrochemical reaction is taken place on the sample area 121 by the analyte, e.g., glucose in the blood sample, reacting with enzyme in the reagent 13. The variation of the current caused by the electrochemical reaction is transmitted to an exterior electric measuring device (not shown in the drawing) by the electrode 12 to quantify the concentration of glucose in blood sample.

In the embodiments according to the present invention, a metallic catalyst is deposited on the surface of the electrode and the metallic catalyst includes, but not limited to, platinum, gold, silver, palladium, ruthenium, rhodium, iridium, oxides or alloys thereof. The presence of the catalyst decreases the activation energy and simultaneously enhances the kinetics of the electrochemical reaction chosen to detect the analyte. In terms of the practical applications, the metallic catalyst will shorten the reaction time and lower the applied electrochemical potential for various electrochemical based detection methods. Lowering the applied potential often leads to the minimization of electrochemical oxidation or reduction of other species presented, resulting in a minimization of interference caused by the unwanted reaction of the confounding species. As a result, a highly specific biosensor can be obtained and produced.

The incorporation of the catalyst and the electrode 12 disposed on the substrate 11 can be accomplished by various manufacturing means including thin film and thick film processes. The thin film process includes physical or chemical vapor deposition. The thick film process includes screen printing, ink-jet printing or other like. In the thick film process, the catalyst can be deposited by combining into a conductive composition for printing the electrode. The conductive composition includes, but not limited to, metallized graphite, polymer and inorganic solvent. The conductive composition will be formulated as an ink or paste that will be suitable for screen or ink-jet printing. The ratios of metallized graphite, polymer and inorganic solvent in the conductive composition can be selected based on the selected deposition technique. In the embodiment according to the present invention, the conductive composition includes:

1. metallized graphite (metal-containing carbon, any ratio of metal deposited on carbon/graphite powder, e.g., 5% Ir from E-TEK) whose composed ratio is in the range of 5-30% in weight;

2. polymer, having a binder and a water-strength polymer, in the range of 5-20% in weight; and

3. water or inorganic solvent, such as 0.1 M, pH 7.0 phosphate buffer.

The metal in the metallized graphite can be pure metal such as platinum, gold, silver, palladium, ruthenium, rhodium, iridium, oxide or alloys thereof. Preferably, the metallized graphite includes metal coated on the surface of the graphite particle with nano-sized dimension.

The binder includes hydroxyethyl cellulose or hydroxypropyl cellulose. The wet-strength polymer includes polyethylenimine; poly(acrylic acid), potassium salt; poly(acrylic acid), sodium salt; poly(acrylic acid-co-acrylamide), potassium salt; poly(acrylic acid), sodium salt-graft-poly(ethylene oxide); poly(2-hydroxyethyl methacrylate); poly(2-hydroxypropyl methacrylate); poly(isobutylene-co-maleic acid) or combinations thereof. Generally, the binder is cross-linked with the wet-strength polymer. Therefore, after forming the electrode on the strip by screen printing, the water or inorganic solvent is evaporated by the following-up steps. The electrode formed by the conductive composition can prevent to be destroyed by the analyte in aqueous solution, for example glucose in blood sample, since the affinity of the chemical structures of the binder cross-linked with the wet-strength polymer has polymeric networks in which they swell rather than be dissolved.

The electrode formed by the conductive composition includes metal in the range of 0.1-5% in weight; graphite in the range of below 55% in weight; and polymer. The difference of the composition between the electrode and the conductive composition is in that the water or organic solvent has been evaporated during the manufacturing process. In the electrode, the graphite is fine particle size and the metal is coated on the surface of the graphite particle with nano-sized dimension.

The strip 10 further includes a conductive layer 14 disposed between the electrode 12 and the substrate 11. The conductive layer 14 is made of graphite dispersed in organic solvent and formed by screen printing, thin film or thick film process. In the embodiment according to the present invention, the conductive layer 14 is disposed on the surface of the substrate 11 and the electrode 12 is disposed on the conductive layer 14 by screen printing, sequentially. That is, the conductive layer 14 and the electrode 12 are formed by screen printing in different steps. The adhesion of the electrode 12 and the substrate 11 can be maintained by the conductive layer 14 disposed therebetween. The area of the electrode 12 is smaller than or equal to that of the conductive layer 14.

In the embodiment according to the present invention, the sample area 121 defined by the electrode 12 is covered by the reagent 13. Again referring to FIG. 1, the strip 10 further includes an insulator 15 covering the electrode 12 except the sample area 121. That is, the insulator 15 includes a concave 151 corresponding to the sample area 121 so that the sample area 121 is not covered by the insulator 15. The reagent 13, including enzyme, is disposed in the concave 151 and covers the sample area 121. The strip 10 further includes a cover 16 which can form a cell (not shown in the drawing) with the concave 151 and the substrate 11. The blood sample entered to the chamber can react with the reagent 13.

The second feature of the present invention is the integration of a heating element into the strip. The integration of this heating element permits the biosensor to operate at an elevated or steady temperature so that the reaction kinetics and faster response can be enhanced. The enhancement can be separately effected on the electrochemical reaction and enzymatic reaction. Cottrell equation describes that the current is a function of time of electrochemical reaction as follows:

$$I ( t ) = \frac{{nFAD}^{1 / 2} {c}_{∞}}{{( π t )}^{1 / 2}} \qquad ( 1 )$$

where n is number of electrons transferred, F is Faraday constant, A is electrode area, and D is diffusion coefficient. Current is directly proportional to the square root of diffusion coefficient. Stokes-Einstein relation further describes that diffusion coefficient is directly proportional to the temperature as follows:

$$D = \frac{{k}_{B} T}{6 π η r} \qquad ( 2 )$$

where k_{B} is Boltzmann constant, η is solution viscosity, and r is the radius of the solvated ion. In consequence, the current along with a rise of temperature causes a diffusion coefficient enhancement of the electrochemical activity.

On the other hand, the rate constant of enzymatic reaction generally increases along with a rise of temperature but not up to the enzyme denaturation temperature. Consequently, rising the temperature must enhance enzymatic reaction to increase the sensitivity of specific detection.

The strip 10 further includes a heating element disposed on the substrate 11 and corresponding to the sample area 121. Again referring to FIG. 1, the heating element includes a first conductor 17, a second conductor 18 and a resistance layer 19 connected to the first and second conductors 17, 18 and corresponding to the sample area 121. In this embodiment, the resistance layer 19 is made of positive temperature coefficient material so that the sample area 121 can be heated by current applied to the first and second conductors 17, 18.

As shown in FIG. 1, the electrode 12 and the heating element are disposed on opposite surfaces of the substrate 11, respectively. Furthermore, the first conductor 17 includes a plurality of first terminals 171 and the second conductor 18 includes a plurality of second terminals 181. The first and second terminals 171, 181 are alternately staggered and connected to the resistance layer 19.

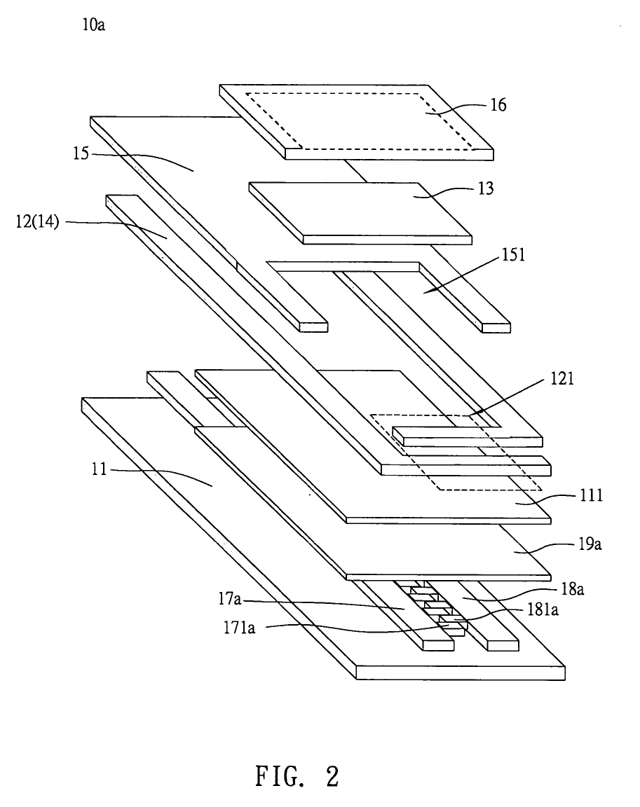

Another embodiment shown in FIG. 2 is the variation of the strip shown in FIG. 1. The difference is in that the strip 10a includes a heating element having a first conductor 17a, a second conductor 18a, and a resistance layer 19a. Similar to the FIG. 1, the first conductor 17a includes a plurality of first terminals 171a and the second conductor 18a includes a plurality of second terminals 181a. The first and second terminals 171a, 181a are alternately staggered and connected to the resistance layer 19a. The difference is in that an insulation layer 111 is disposed between the electrode 12 and the resistance layer 19a since the heating element and the electrodes 12 are disposed on the same surface of the substrate 11.

This integrated heating element can be produced in many ways, including, but not limited to the thick film printing of a positive-temperature coefficient material. The incorporation of this heating element permits the biosensor to operate at a selected or preferred temperature, for example, 25° C. to 60° C. or 30 to 45° C. This enhancement of the performance of the biosensor can be realized, but not limited to, any enzymatic based biosensors. For example, the operation of a biosensor, such as the strip of the blood glucose sensor can be improved by the integration of a heating element. This is because the enzymatic reaction of the oxidation of glucose at 37° C. is improved, compared with that carried out at ambient temperature.

Again referring to FIG. 1, the resistance layer 19 with flexibility is made of positive temperature coefficient (PTC) material so that heat generated by applying current to the conductors 17 and 18 can be transmitted to the sample area 121. The PTC material can be formed by thick film process so that the first and second terminals 171, 181 can be covered and connected by the resistance layer 19.

An embodiment of a biosensor of the present invention is a disposable type of glucose sensor used by diabetics around the world based on an enzymatic reaction. Generally, the level of the blood sugar of diabetics is measured based on an enzymatic reaction as the following chemical equation:

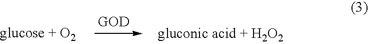

where GOD is glucose oxidase.

Hydrogen peroxide, H₂O₂, is an electrochemically active species that can be either oxidized or reduced under appropriate conditions. When blood, physiological fluid or other media encounters the enzyme in the reagent, the current produced from the electrochemical oxidation or reduction of H₂O₂ or NADH can be quantified by using chronoamperometry or similar current measuring technique. Furthermore, the biosensor of the present invention is able to quantify H₂O₂ which is produced from a variety of enzymatic reaction. In the embodiment of the present invention, the reagent 13 contains the oxidoreductase such as, but not limited to, glucose oxidase, glucose dehydrogenase, cholesterol oxidase, D-3-hydroxybutyrate dehydrogenase, fructosyl amino acid oxidase, or combinations thereof.

The embodiment according to the present invention further includes an electric measuring device having a slot enabling the strip 10 or 10a to insert therein.

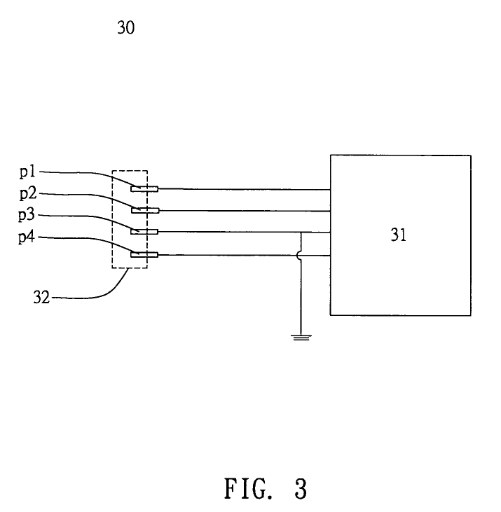

As shown in FIG. 3, an electric measuring device 30 includes a microcontroller unit 31 and four pins p1, p2, p3 and p4 connected to the microcontroller unit 31, respectively. In practice, the four pins p1, p2, p3 and p4 are disposed in a slot 32 enabling the strip 10 or 10a to insert therein. The four pins p1, p2, p3 and p4 are disposed in the slot 32 are corresponding to the electrodes and the conductors of the strip so that the microcontroller unit 31 can be electrically connected to the electrodes and the conductors, respectively. When the strip 10a inserted to the slot 32, the pins p1 and p4 are connected to the two electrodes 12, respectively, and the pins p2 and p3 are connected to the first and second conductors 17a, 18a, respectively. Since the first and second conductors 17a, 18a are connected by the resistance layer 19a so that the pins p2 and p3 are electrically connected by the conductors. The microcontroller unit 31 can recognize that the strip 10a is inserted to the slot 32 by the electrical connection of the pins p2 and p3 and start to measure current variation from the pins p1 and p4 for detecting the electrochemical reaction. The two conductors 17a and 18a on the strip 10a can be a key to start the electrical measuring device 30.

In the same way, the strip 10 can also be a key to start the electrical measuring device 30, as long as the pins p1, p2, p3 and p4 in the slot 32 are corresponding to the electrodes 12 and the conductors 17 and 18, respectively.

To sum up, while the invention has been described by way of example and in terms of preferred embodiments, it is to be understood that the invention is not limited to the disclosed embodiments. To the contrary, it is intended to cover various modifications and similar arrangements as would be apparent to those skilled in the Art. Therefore, the scope of the appended claims should be accorded the broadest interpretation so as to encompass all such modifications and similar arrangements.

What I claimed is:

1. A biosensor comprising:
a strip having:
a substrate;
at least two electrodes disposed on the substrate to define a sample area; and
a reagent disposed on the sample area,
wherein the electrode having:
metal in the range of 0.1-5% in weight;
graphite in the range of below 55% in weight; and
polymer.

2. The biosensor as recited in claim 1, wherein the graphite is fine particle size and the metal is coated on the surface of the graphite particle with nano-sized dimension.

3. The biosensor as recited in claim 1, wherein the metal comprises platinum, gold, silver, palladium, ruthenium, rhodium, iridium, oxides or alloys thereof.

4. The biosensor as recited in claim 1, wherein the polymer comprises a binder and a wet-strength polymer.

5. The biosensor as recited in claim 4, wherein the binder comprises hydroxyethyl cellulose and hydroxypropyl cellulose.

6. The biosensor as recited in claim 4, wherein the wet-strength polymer comprises polyethylenimine; poly(acrylic acid), potassium salt; poly(acrylic acid), sodium salt; poly(acrylic acid-co-acrylamide), potassium salt; poly(acrylic acid), sodium salt-graft-poly(ethylene oxide); poly(2-hydroxyethyl methacrylate); poly(2-hydroxypropyl methacrylate); poly(isobutylene-co-maleic acid) or combinations thereof.

7. The biosensor as recited in claim 1, further comprising a conductive layer disposed between the electrode and the substrate.

8. The biosensor as recited in claim 7, wherein the conductive layer is screen-printed by graphite dispersed in an organic solvent.

9. The biosensor as in claim 7, wherein the conductive layer and the electrode are formed by screen printing in different steps.

10. The biosensor as recited in claim 1, wherein the reagent comprises enzyme.

11. The biosensor as recited in claim 10, wherein the enzyme comprises oxidoreductase.

12. The biosensor as recited in claim 11, wherein the oxidoreductase is glucose oxidase, glucose dehydrogenase, cholesterol oxidase, D-3-hydroxybutyrate dehydrogenase, fructosyl amino acid oxidase, or combinations thereof.

13. A biosensor comprising:
a strip having:
a substrate;
at least two electrodes disposed on the substrate to define an sample area;
a reagent disposed on the sample area; and
a heating element disposed on the substrate and corresponding to the sample area.

14. The biosensor as recited in claim 13, wherein the heating element comprises a first conductor, a second conductor and a resistance layer connected to the first and second conductors.

15. The biosensor as recited in claim 14, wherein the resistance layer is made of positive temperature coefficient material.

16. The biosensor as recited in claim 14, wherein the first conductor has a plurality of first terminals, the second has a plurality of second terminals, and the first and second terminals are alternately staggered therebetween and connected to the resistance layer.

17. The biosensor as recited in claim 13, wherein the sample area is heated by the heating element to an elevated temperature.

18. The biosensor as recited in claim 17, wherein the elevated temperature is at a range of 25 to 60° C. or 30 to 45° C.

19. The biosensor as recited in claim 13, wherein the sample area is heated by the heating element to a steady temperature.

20. The biosensor as recited in claim 19, wherein the steady temperature is 37° C.

21. The biosensor as recited in claim 13, wherein the two electrodes and the heating element are disposed on opposite surfaces of the substrate, respectively.

22. The biosensor as recited in claim 13, further comprising an insulation layer disposed between the heating element and the sample area.

23. The biosensor as recited in claim 13, further comprising an electric measuring device having a slot enabling the strip to insert therein.

24. The biosensor as recited in claim 23, further comprising pins disposed in the slot and corresponding to the two electrodes and the first and second conductors.

25. The biosensor as recited in claim 24, wherein the electric measuring device is capable for recognizing the strip inserted to the slot by electrical connection of the pins and the first and second conductors, respectively.

26. A biosensor comprising:
a strip having:
a substrate;
at least two electrodes disposed on the substrate to define an sample area;
a reagent disposed on the sample area; and
a heating element disposed corresponding to the sample area,
wherein the electrode comprises metal in the range of 0.1-5% in weight; graphite in the range of below 55% in weight; and polymer.

27. The biosensor as recited in claim 26, further comprising an electric measuring device having a slot enabling the strip to insert therein.

28. The biosensor as recited in claim 27, further comprising pins disposed in the slot and corresponding to the two electrodes and the first and second conductors so that the electric measuring device is capable for recognizing the strip inserted to the slot by electrical connection of the pins and the first and second conductors, respectively.

29. A conductive composition comprising:
metallized graphite in the range of 5-30% in weight; and
binder and wet-strength polymer in the range of 5-20% in weight.

30. The conductive composition as recited in claim 29, wherein the metallized graphite comprises metal coated on the surface of graphite particle with nano-sized dimension.

31. The conductive composition as recited in claim 30, wherein the metal comprises platinum, gold, silver, palladium, ruthenium, rhodium, iridium, oxides or alloys thereof.

32. The conductive composition as recited in claim 29, wherein the binder comprises hydroxyethyl cellulose or hydroxypropyl cellulose.

33. The conductive composition as recited in claim 29, wherein the wet-strength polymer comprises polyethylenimine; poly(acrylic acid), potassium salt; poly(acrylic acid), sodium salt; poly(acrylic acid-co-acrylamide) potassium salt; poly(acrylic acid), sodium salt-graft-poly(ethylene oxide); poly(2-hydroxyethyl methacrylate); poly(2-hydroxypropyl methacrylate); poly(isobutylene-co-maleic acid) or combinations thereof.

34. The conductive composition as recited in claim 30, further comprising inorganic solvent.
